(19) United States
(12) Patent Application Publication
(10) Pub. No.: US 2010/0274050 A1
(43) Pub. Date: Oct. 28, 2010

(54) SOLID MILNACIPRAN AND PROCESS FOR THE PREPARATION OF THE SAME

(75) Inventors: Joseph Prabahar KOILPILLAI, Navi Mumbai (IN); Pravin Chhaburao THOMBRE, Dombivali(East) (IN); Sudam Nanabhau SINARE, Navi Mumbai (IN); Mubeen Ahmed KHAN, Navi Mumbai (IN)

Correspondence Address: GLENMARK GENERICS INC., 750 CORPORATE DRIVE, MAHWAH, NJ, 07430, US

(73) Assignee: Glenmark Generics Limited, Mumbai (IN)

(21) Appl. No.: 12/497,823

(22) Filed: Jul. 6, 2009

(30) Foreign Application Priority Data
Apr. 23, 2009 (IN) 1086/MUM/2009

(51) Int. Cl. C07C 237/24; C07C 231/12

(52) U.S. Cl. 564/164

(57) ABSTRACT

The present invention provides novel solid milnacipran in crystalline form-G and a process for its preparation. The present invention also provides a process for the preparation of milnacipran hydrochloride from the novel solid crystalline milnacipran.

### PRIORITY

This application claims the benefit under 35 U.S.C. §119 to Indian Provisional Application 1086/MUM/2009, filed on Apr. 23, 2009, the contents of which is incorporated by reference herein.

### BACKGROUND OF THE INVENTION

1. Technical Field

The present invention generally relates to a solid crystalline milnacipran and/or pharmaceutically acceptable salts thereof, processes for their preparation; and pharmaceutical compositions containing the same.

2. Description of the Related Art

Milnacipran, also known as (±)-[1R(S), 2S(R)]-2-(aminomethyl)-N,N-diethyl-1-phenylcyclopropanecarboxamide, is represented by the structure of formula I:

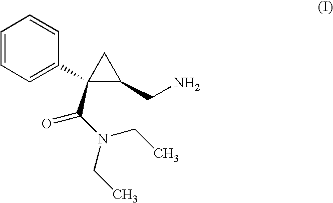

Milnacipran hydrochloride is a selective norepinephrine and serotonin reuptake inhibitor, it inhibits norepinephrine uptake with greater potency than serotonin and it is useful in the treatment of depression and chronic pain conditions like fibromyalgia syndrome and lupus. Milnacipran hydrochloride is marketed in the United States under the brand name Savella® in the form of 12.5, 25, 50 and 100 mg tablets for fibromyalgia syndrome; while in Europe, it is available under the brand name Ixel® in the form of 25, 50 and 100 mg tablets for depression.

U.S. Pat. No. 4,478,836 (“the '836 patent”) discloses milnacipran and its hydrochloride salt form. The '836 patent discloses a process for the preparation of milnacipran hydrochloride by the reaction of an acid chloride of 1-phenyl 1-ethoxycarbonyl 2-aminomethyl cyclopropane (Z) with diethylamine and salifying with hydrochloric acid. The '836 patent however, does not describe whether the milnacipran so obtained is solid. The entirety of the '836 patent is incorporated herein by reference.

Patent Publication WO2008/104957 (the '957 publication) discloses a multistep process for the preparation of milnacipran hydrochloride by the reaction of (Z)-1-phenyl-1-diethylaminocarbonyl-2-phthalimidomethyl cyclopropane of formula II with hydrazine hydrate and the resultant milnacipran product isolated as a wet compound with a significant amount of phthalimide by-products, which are removed by acid-base treatment and whereupon the milnacipran is directly converted to its hydrochloride salt form. The process disclosed in the '957 publication is schematically represented by scheme I:

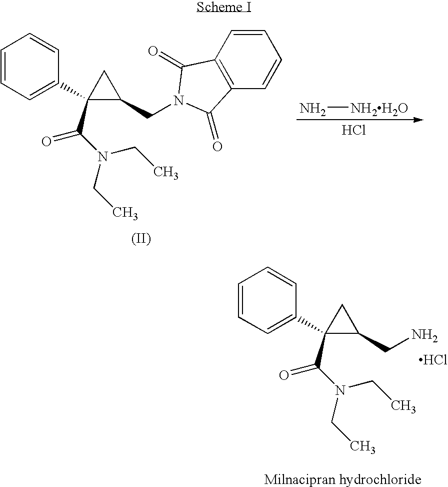

### SUMMARY OF THE INVENTION

The present invention provides milnacipran in solid form.

The present invention provides the solid milnacipran in crystalline form.

The present invention further provides the solid crystalline milnacipran designated as form-G and hereinafter referred by this designation.

The present invention further provides a process for the preparation of solid crystalline milnacipran form-G, comprising;
a) reacting (Z)-1-phenyl-1-diethylaminocarbonyl-2-phthalimidomethyl cyclopropane of formula II with monomethylamine in a first organic solvent to form a reaction solution,
b) isolating the milnacipran from the reaction solution.
c) crystallizing the milnacipran in solid form with a second organic solvent.
   wherein the second organic solvent selected from C₁₋₄ alcohols, ketones, esters, hydrocarbons, ethers, halogenated solvents, water and their mixtures.

The present invention provides pharmaceutically acceptable salt of solid crystalline milnacipran form-G.

The present invention provides a process for the preparation of a pharmaceutically acceptable salt of solid crystalline milnacipran form-G, comprising;
a) reacting (Z)-1-phenyl-1-diethylaminocarbonyl-2-phthalimidomethyl cyclopropane of formula II with monomethylamine in a first organic solvent to form a reaction solution,
b) isolating the milnacipran from the reaction solution,
c) crystallizing the milnacipran in solid form with a second organic solvent,
d) converting the milnacipran into its pharmaceutically acceptable salts thereof.
   wherein the second organic solvent selected from C₁₋₄ alcohols, ketones, esters, hydrocarbons, ethers, halogenated solvents, water and their mixtures.

The present invention provides essentially pure milnacipran in solid form.

The present invention further provides milnacipran in solid form, which has a purity of at least about 97% by HPLC.

The present invention further provides milnacipran in solid form, which has a purity of at least about 98% by HPLC.

The present invention further provides milnacipran in solid form, which has a purity of at least about 99% by HPLC.

The present invention further provides milnacipran in solid form, which has a purity of at least about 99.5% by HPLC.

The present invention further provides milnacipran in solid form, which has a purity of at least about 99.8% by HPLC.

The present invention further provides solid crystalline milnacipran form-G.

The present invention provides solid crystalline milnacipran in form-G characterized by an X-ray powder diffraction pattern (XRPD), which is substantially in accordance with FIG. 1.

The present invention provides solid crystalline milnacipran form-G characterized by a differential scanning calorimetry (DSC) thermogram, which is substantially in accordance with FIG. 2.

The present invention provides solid crystalline milnacipran in form-G characterized by a thermo gravimetric analysis (TGA), which is substantially in accordance with FIG. 3.

The present invention provides a pharmaceutical composition comprising a therapeutically effective amount of a milnacipran form-G and at least one pharmaceutically acceptable carrier.

### BRIEF DESCRIPTION OF THE DRAWINGS

FIG. 1 is a characteristic XRPD of solid crystalline milnacipran form-G.

FIG. 2 is a characteristic DSC thermogram of solid crystalline milnacipran form-G.

FIG. 3 is a characteristic TGA scan of solid crystalline milnacipran form-G.

FIG. 4 is a characteristic XRPD of milnacipran hydrochloride.

FIG. 5 is a characteristic DSC thermogram of milnacipran hydrochloride.

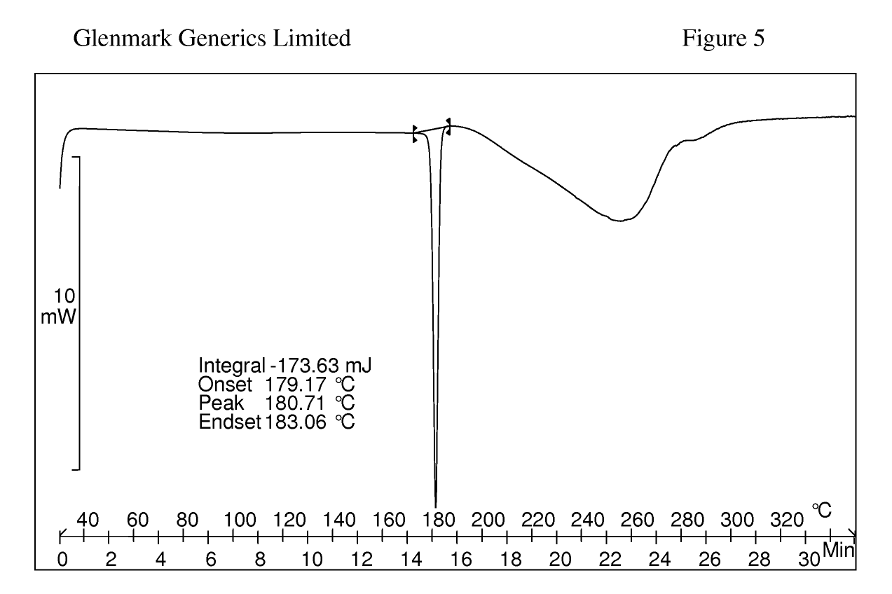

FIG. 6 is a characteristic TGA scan of milnacipran hydrochloride.

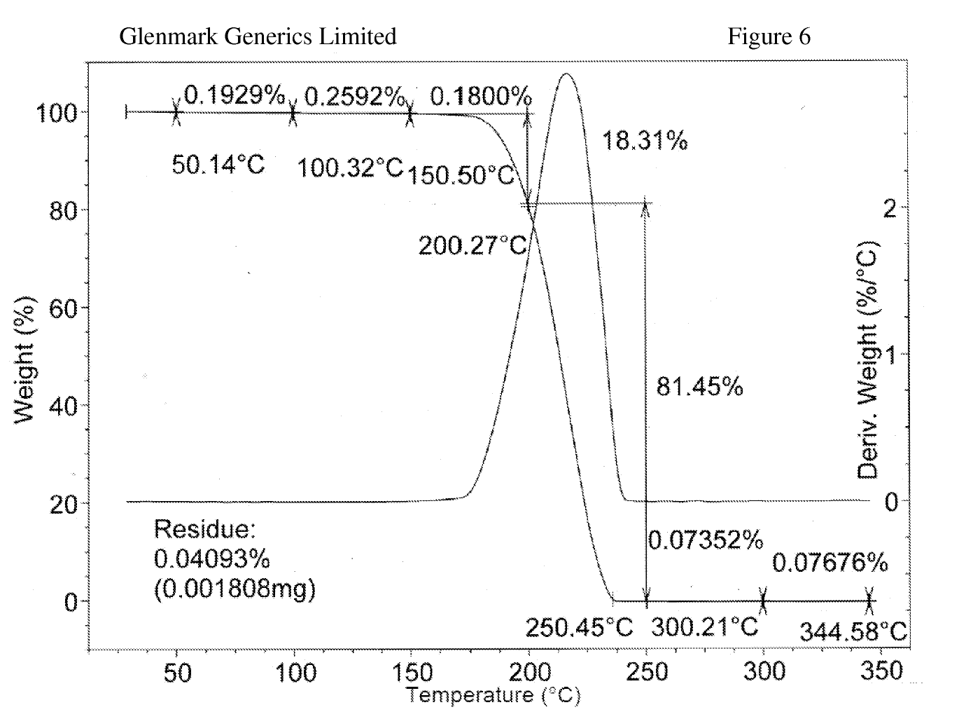

FIG. 7 is a characteristic scanning electron micrograph (SEM) of milnacipran hydrochloride.

FIG. 8 is a characteristic nuclear magnetic resonance (NMR) spectra of solid crystalline milnacipran form-G.

### DETAILED DESCRIPTION OF THE INVENTION

The present invention provides solid milnacipran in crystalline form, designated as form-G or pharmaceutically acceptable salts thereof and processes for their preparation; and pharmaceutical compositions comprising the same.

The available processes for the preparation of milnacipran do not effectuate to a desirable form of milnacipran, i.e., an isolated form. It would be a prodigious contribution to the arts to have both a convenient and cost efficient process for preparing milnacipran in solid form and a process for its conversion into its corresponding pharmaceutically acceptable salts thereof, which are commercially scalable. The present invention provides a novel process for isolating milnacipran in solid form. The milnacipran in solid form is in the crystalline form, with a purity of at least about 99.5%, as measured by high performance liquid chromatography (HPLC).

The present invention provides a process for the preparation of solid crystalline milnacipran, or a pharmaceutically acceptable salt thereof comprising:
a) reacting the (Z)-1-phenyl-1-diethylaminocarbonyl-2-phthalimidomethyl cyclopropane of formula II with monomethylamine in a first organic solvent to form a reaction solution,
b) isolating the milnacipran from the reaction solution,
c) crystallizing the milnacipran in solid form with a second organic solvent.

The present invention provides the solid crystalline milnacipran as form-G optionally may be converted into a pharmaceutically acceptable salt.

(Z)-1-Phenyl-1-diethylaminocarbonyl-2-phthalimidomethylcyclopropane of formula II is a known compound and can be produced by methods known and recognized by the organic chemist of ordinary skill in the art. For example, such a process is described in EP0200638, which is included by reference herein in its entirety.

The reaction of (Z)-1-phenyl-1-diethylaminocarbonyl-2-phthalimidomethyl cyclopropane of formula II with monomethylamine is carried out in a first organic solvent. The (Z)-1-phenyl-1-diethylaminocarbonyl-2-phthalimidomethyl cyclopropane may be dissolved in the first organic solvent at a temperature suitable for its complete dissolution, such as at about ambient temperature to about the reflux temperature of the solvent used. Preferably, at a temperature ranging from about 30° C. to about 60° C.

The first organic solvent is selected from C₁₋₄ alcohols such as methanol, ethanol, isopropanol, n-propanol, butanol, isobutanol and the like; hydrocarbons such as n-hexane, n-heptane, n-pentane, cyclohexane, benzene, toluene and the like; halogenated solvents such as dichloromethane, dichloroethane, chloroform, carbon tetrachloride and the like; esters such as methyl acetate, ethyl acetate, isopropyl acetate, tertiary butyl acetate and the like, water and their mixtures. Preferably the first organic solvent is selected from methanol, ethanol, isopropanol, ethyl acetate, isopropyl acetate, toluene, dichloromethane, water and mixtures thereof; more preferably the first organic solvent is methanol, ethanol, toluene, ethyl acetate, water and their mixtures; still more preferably the first organic solvent is a mixture of toluene and water.

The monomethylamine can either be a solution in water or in the first organic solvent. The methylamine concentration may range from about 1% by weight to about 40% by weight, preferably about 10% by weight to about 40% by weight.

The order of addition of compound of formula II with monomethylamine may follow either the addition of (Z)-1-phenyl-1-diethylaminocarbonyl-2-phthalimidomethyl cyclopropane of formula II into monomethylamine solution obtained or the addition of monomethylamine solution into (Z)-1-phenyl-1-diethylaminocarbonyl-2-phthalimidomethyl cyclopropane of formula II.

The reaction temperature should be sufficient to effect the reaction. Typically the reaction temperature may be from about 5° C. to about 80° C. Preferably the reaction temperature is about 20° C. to about 50° C., more preferably at about 25° C. to about 35° C. The reaction time is usually about 2 hours to about 30 hours, preferably about 15 hours to about 25 hours.

After completion of the reaction, a bilayer is formed where N,N′-dimethylphthalamide, which is a by-product may be present under dissolved conditions in the aqueous layer. The facile removal of N,N′-dimethylphthalamide can be undertaken by separating the aqueous layer from the reaction mixture by any phase separation method known in the art. The resultant organic layer containing the milnacipran may be extracted with water under acidic conditions by adjusting the pH about 2 to about 4, wherein the adjustment of pH may be carried out with acids known in the art, for example hydrochloric acid, acetic acid and the like, preferably the acid is hydrochloric acid. Further, one skilled in organic synthesis would recognize a and appreciate separating from the organic layer any unwanted starting material (Z)-1-phenyl-1-diethylaminocarbonyl-2-phthalimidomethyl cyclopropane of formula II by a phase separation method known in the art.

The resultant water containing the milnacipran product may be extracted with an organic solvent under basic conditions by adjusting the pH from about 10 to about 12, wherein the organic solvent may be selected from halogenated solvents such as dichloromethane, dichloroethane, chloroform, carbon tetrachloride and the like, hydrocarbons such as n-hexane, n-heptane, cyclohexane, benzene, toluene and the like; esters such as methyl acetate, ethyl acetate, isopropyl acetate, tertiary butyl acetate and the like; and mixtures thereof. Preferably the organic solvent is selected from dichloromethane, chloroform, toluene, ethyl acetate and a mixture thereof, more preferably the organic solvent is dichloromethane or toluene.

The adjustment of pH may be carried out with bases, wherein the bases are known in the art, for example sodium hydroxide, potassium hydroxide and the like, preferably the base is sodium hydroxide. The resultant organic solvent containing milnacipran may be concentrated under vacuum to get the residue by any method known in the art, for example distillation, evaporation, rotational drying (such as with the Buchi Rotavapor), freeze-drying, fluidized bed drying, flash drying, spin flash drying, and the like.

The resultant residue may be dissolved in a second organic solvent. The second organic solvent may includes, but is not limited to an alcohol such as C₁₋₄ alcohol selected from methanol, ethanol, isopropanol, n-propanol, butanol, isobutanol and the like; ketone such as acetone, ethyl methyl ketone, methyl isobutyl ketone, cyclohexanone and the like; ester such as methyl acetate, ethyl acetate, isopropyl acetate, tertiary butyl acetate and the like; hydrocarbon such as n-pentane, n-hexane, n-heptane, cyclohexane, toluene and the like; ether such as isopropyl ether, diethyl ether, tetrahydrofuran, methyl tertiary butyl ether, 1,4-dioxane and the like; halogenated solvent such as dichloromethane, dichloroethane, chloroform, carbon tetrachloride and the like; water and their mixtures. Preferably the second organic solvent is selected from methanol, ethanol, isopropanol, acetone, ethyl acetate, isopropyl acetate, isopropyl ether, toluene, cyclohexane, n-pentane, n-hexane, n-heptane, dichloromethane, water and mixtures thereof. More preferably, the second organic solvent is n-pentane, n-heptane, cyclohexane, isopropyl ether and mixtures thereof.

Then, the solution may be heated to dissolve the milnacipran. The temperature suitable for dissolving milnacipran depends on the solvent used and the amount of milnacipran in the solution. Typically, the solution may be heated at a temperature of at least about 30° C. to about 50° C. Preferably, the solution may be heated at about 40° C. to about 45° C.

The isolation of the resultant milnacipran in solid form from the organic layer can be accomplished in any method known in the art. These isolation techniques include crystallization, solvent precipitation, concentration by subjecting the solution to heating, spray drying, freeze drying, evaporation on rotary evaporator under vacuum, agitated thin film evaporator (ATFE) and the like. In like manner, the milnacipran in solid form can be recovered by any conventional technique known in the art, for example filtration. Typically, if stirring is involved, the temperature during stirring can range from about −10° C. to about +25° C., preferably from about 0° C. to about +15° C., more preferably from about 5° C. to about 10° C.

The resultant product optionally may be further dried. Drying can be suitably carried out in a tray dryer, vacuum oven, air oven, fluidized bed drier, spin flash dryer, flash dryer and the like. The drying can be carried out at a temperature ranging from about 30° C. to about 40° C. The drying can be carried out a time period ranging from about 1 hour to about 20 hours, preferably about 5 hours.

The present invention provides a solid crystalline milnacipran form-G or pharmaceutically acceptable salts thereof, prepared in the process described herein

A high purity level of the resulting milnacipran in solid form, obtained by the aforementioned process, may have a chemical purity, as measured by HPLC, of at least about 97%, preferably at least about 98% more preferably at least about 99%, still more preferably at least about 99.5%, most preferably at least about 99.8%.

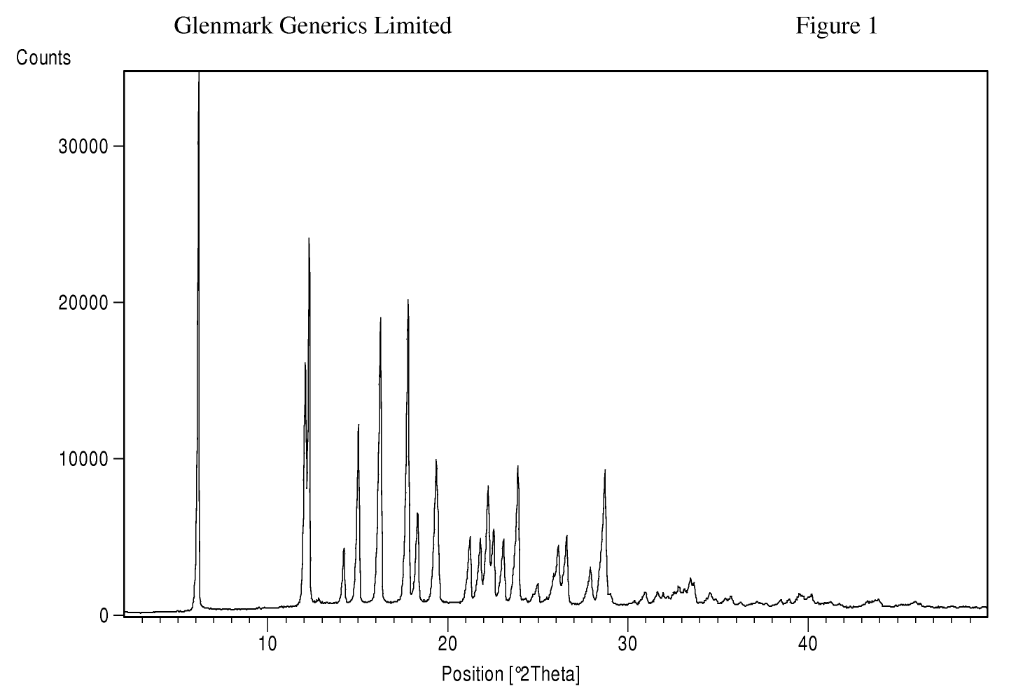

The present invention provides characterization of a milnacipran in solid crystalline form-G via X-ray powder diffraction pattern and/or melting point. The X-Ray powder diffraction pattern, which is substantially characterized in FIG. 1, can be measured by an X-ray powder Diffractometer equipped with a Cu-anode (λ=1.54 Angstrom), X-ray source operated at 45 kV, 40 mA and a Ni filter is used to strip K-beta radiation. Two-theta calibration is performed using an NIST SRM 640c Si standard. The sample was analyzed using the following instrument parameters: measuring range=2-50° 2θ; step width=0.017°; and measuring time per step=5 sec.

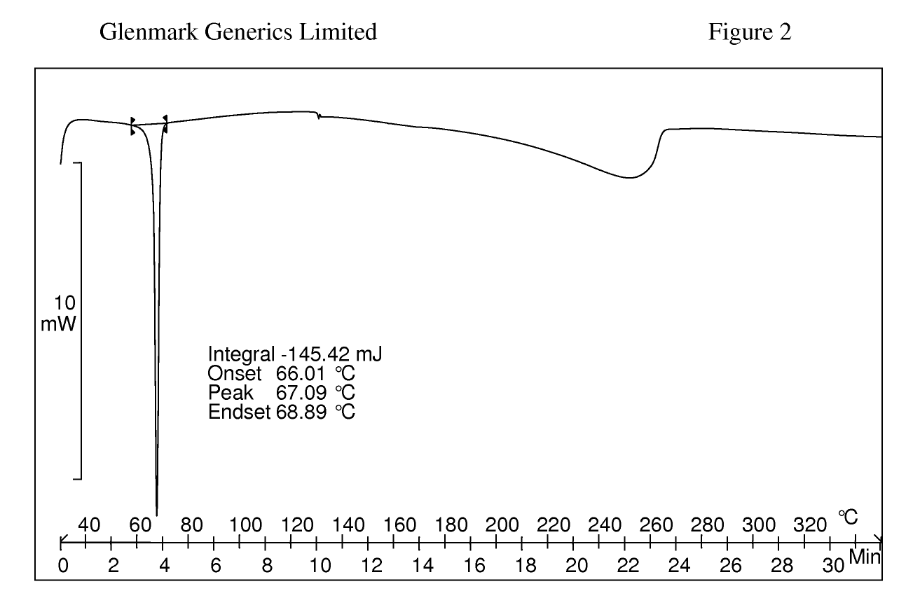

The present invention further provides a milnacipran in solid crystalline form-G, with a differential scanning calorimetry thermogram, which is substantially characterized in FIG. 2, is measured by a Differential Scanning Calorimeter (DSC 822, Mettler Toledo) at a scan rate of 10° C. per minute with an Indium standard. Milnacipran in solid crystalline form-G exhibits a predominant endotherm peak at about 67° C. Whereupon, the endotherm measured by a particular differential scanning calorimeter is dependent upon a number of factors, including the rate of heating (i.e., scan rate), the calibration standard utilized, instrument calibration, relative humidity, and upon the chemical purity of the sample being tested. Thus, an endotherm as measured by DSC on the instrument identified above may vary by as much as ±1° C. or even ±2° C.

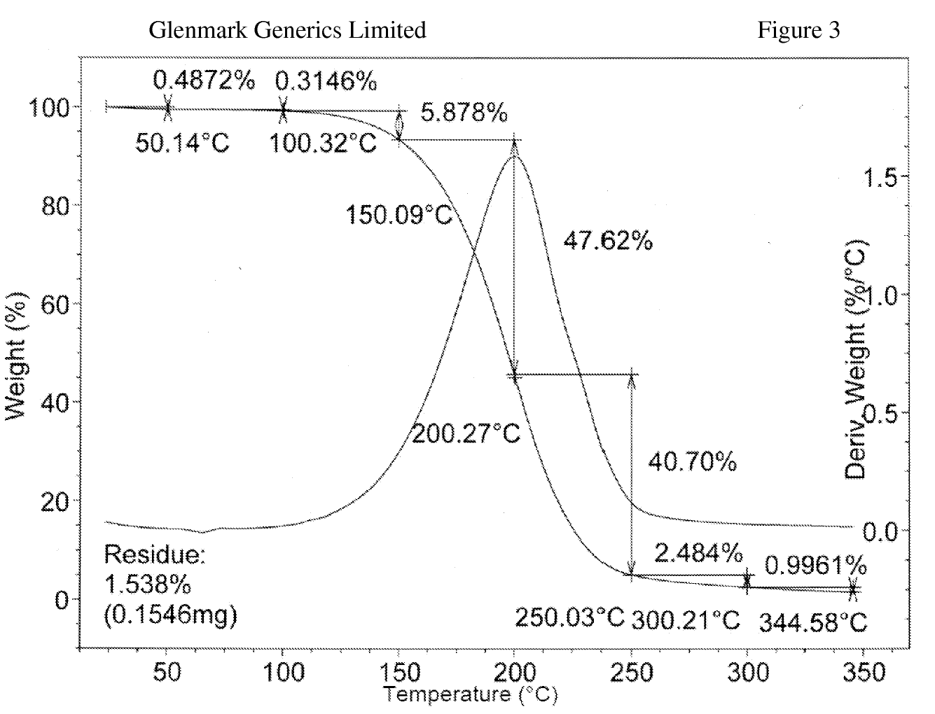

The present invention further provides a milnacipran in solid crystalline form-G, with a thermogravimetric analysis (TGA) scan, which is substantially characterized in FIG. 3, recorded on TGA Q500 V 20.6 in platinum pan with a temperature rise of 10° C./min in the range 30° C. to 350° C.

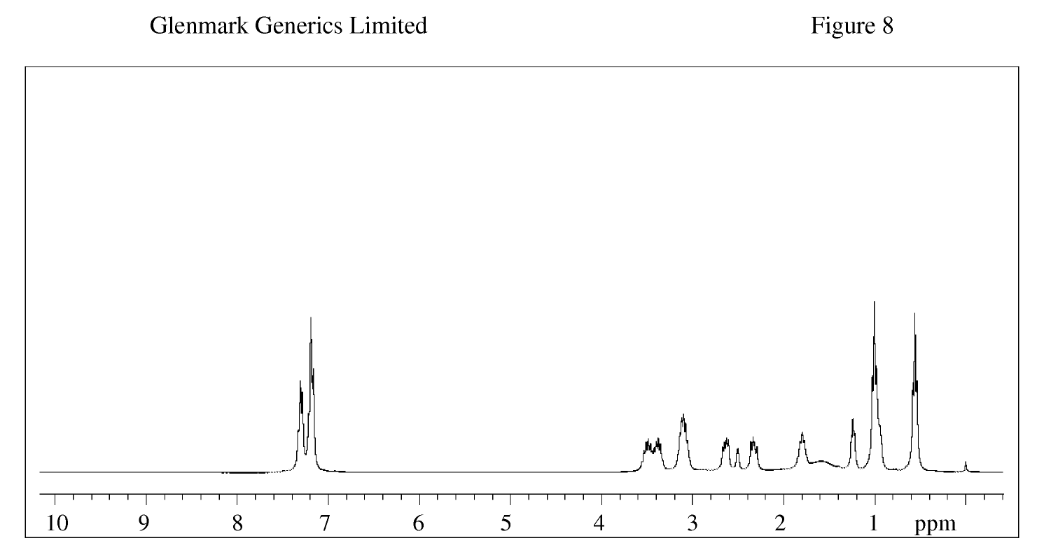

The present invention further provides a milnacipran in solid crystalline form-G, with an NMR scan, which is substantially in accordance with FIG. 8. The NMR determination is performed using 300 MHz Varian NMR Spectrometer; sample preparation: dissolve 5 mg of sample in 0.7 ml DMSO.

The present invention provides pharmaceutically acceptable salts of milnacipran, preferably hydrochloride salt, comprising providing a milnacipran in solid form, prepared by the process described above, as a starting material or as an intermediate, wherein the yield and the purity of the pharmaceutically acceptable salts thereof may have a purity of at least about 99.8% as determined by HPLC.

The present invention further provides a process for a preparation of a pharmaceutically acceptable salt of milnacipran in solid form, preferably milnacipran hydrochloride salt comprising;
a) providing a milnacipran in solid crystalline form, obtained by the process described above, dissolved in one or more organic solvents,
b) heating the solution to completely dissolve the milnacipran in solid form,
c) treating the resultant solution with hydrochloric acid,
d) cooling the resultant reaction solution,
e) isolating the milnacipran hydrochloride.

The one or more organic solvents is selected from, but not limited to, C₁₋₄ alcohol selected from methanol, ethanol, n-propanol, isopropanol, n-butanol, isobutyl alcohol, tertiary butyl alcohol and the like and mixtures thereof, ether selected from tetrahydrofuran, dimethylether, diethyl ether, methylethylether, diisopropylether, methyl tertiary butyl ether and the like; ester such as ethyl acetate, isopropyl acetate, isobutyl acetate, water and mixtures thereof. Preferably the organic solvents are selected from methanol, ethanol, isopropanol, ethyl acetate, tetrahydrofuran. More preferably the organic solvents are isopropanol, ethyl acetate and mixtures thereof.

Typically, heat may be applied to the solution in a) of the process above for complete dissolution of the milnacipran, where the temperature is from at least about 30° C. to about 50° C. Preferably, the solution is heated at about 30° C. to about 40° C.

The hydrochloric acid for the salt formation in c) may be aqueous, anhydrous, gaseous or organic solution, for example aqueous hydrochloric acid or a solvent containing hydrochloric acid or hydrochloric acid gas. Preferably, a solvent containing hydrochloric acid can be used; wherein the solvent is selected from, but not limited to methanol, ethanol, isopropanol, ethyl acetate; more preferably the organic solvent is isopropanol or ethyl acetate. Most preferably, HCl in ethyl acetate.

Temperature for cooling the reaction solution can be carried out from about 30° C. or less, preferably at temperature from about 0° C. to about 20° C. The cooling temperature should be conducive to the isolation of the milnacipran hydrochloride by conventional techniques known in the art, for example filtration. In the event that stirring is involved, the temperature during stirring can range from about 0° C. to about +20° C., preferably at about 10° C. to about 15° C.

The resultant product optionally may be further dried. Drying can be suitably carried out in a tray dryer, vacuum oven, air oven, fluidized bed drier, spin flash dryer, flash dryer and the like. The drying can be carried out at a temperature ranging from about 30° C. to about 70° C., preferably at a temperature ranging from about 40° C. to about 60° C., more preferably at about 45° C. to about 50° C. The drying can be carried out a time period ranging from about 1 hour to about 20 hours, preferably about 5 hours. A high purity level of the resulting milnacipran hydrochloride, obtained by the aforementioned process, may have a chemical purity of at least about 98%, as measured by HPLC, preferably at least about 99.5%, as measured by HPLC, more preferably at least about 99.8%, as measured by HPLC.

The present invention provides a milnacipran hydrochloride, obtained by the process described herein, having a chemical purity, as measured by HPLC, of at least about 98% preferably at least about 99.5%, and more preferably at least about 99.8%; and substantially free of one or more of the following impurities, as determined by HPLC.
i) 1-phenyl-1-diethylaminocarbonyl-2-phthalimidomethyl cyclopropane of formula II;

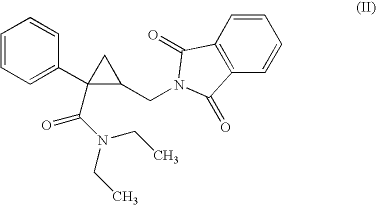

ii) 1-phenyl-1-ethylaminocarbonyl-2-phthalimidomethyl cyclopropane of formula III;

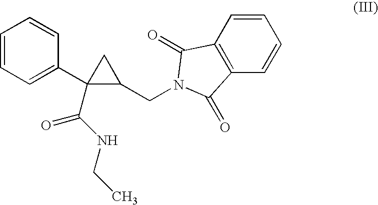

iii) 2-(aminomethyl)-N-ethyl-1-phenylcyclopropanecarboxamide of formula IV;

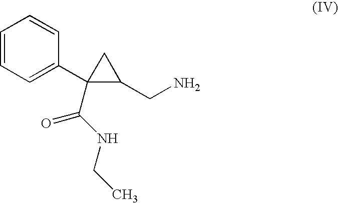

iv) Trans isomer of 2-(aminomethyl)-N,N-diethyl-1-phenylcyclopropane carboxamide (trans milnacipran) of formula V;

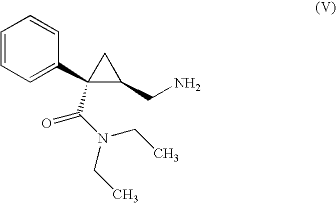

wherein the word “substantially free” refers to milnacipran hydrochloride having less than about 0.1%, of formula-II, or formula-III, or formula-IV or formula-V, as measured by HPLC, more preferably less than about 0.05% of formula-II or formula-III, or formula-IV or formula V, as measured by HPLC.

The high performance liquid chromatography (HPLC) used to analyze solid crystalline milnacipran form-G or pharmaceutically acceptable salts thereof, used conditions as described below:

| | |
|---|---|
| Column: | XTerra RP-18, 250 × 4.6, 5μ |
| Column temperature: | 25° C. |
| Mobile phase: | |
| Mobile phase A: | Triethylamine:ortho phosphoric acid:water |
| | (1:2:1000, v/v/v) |
| Mobile phase B: | Acetonitrile |

| | | |
|---|---|---|
| Time | % Mobile | % Mobile |
| (minutes) | phase A | phase B |
| 0.0 | 80 | 20 |
| 05 | 80 | 20 |
| 40 | 20 | 80 |
| 45 | 20 | 80 |
| 50 | 80 | 20 |
| 60 | 80 | 20 |

| | |
|---|---|
| Diluent: | water:methanol:acetonitrile (50:25:25, v/v/v) |
| Flow rate: | 1.0 mL/minute |
| Detection: | UV 225 nm |
| Injection volume: | 10 μL |

The present invention provides a milnacipran hydrochloride, obtained by the process disclosed herein, is characterized by an average size of about 50 μm for 50% of the particles, about 20 μm for 10% of the particles and about 100 μm for 90% of the particles, obtained upon milling. The present invention further provides milnacipran hydrochloride, obtained by the process disclosed herein, is characterized by an average size of about 40.3 μm for 50% of the particles, about 16.2 μm for 10% of the particles and about 79.3 μm for 90% of the particles, obtained upon milling.

The particle size measurement employed Malvern Mastersizer-2000, equipped with Malvern hydro2000S (A) sample handling unit with conditions described below:

Material RI: 1.60, material absorption: 0.001, dispersant name: liquid paraffin, dispersant RI: 1.468, sensitivity: normal, measurement time: 12 seconds, background time: 12 seconds, obscuration range: 10-20%, stirrer speed: 2500 rpm, ultrasonic: 120 seconds, premeasurement, and tip displacement (sonication): 60%.

The present invention provides a milnacipran hydrochloride, obtained by the process disclosed herein, is characterized by a specific surface area of about 0.1 m²/gm to about 5 m²/gm, as measured by BET method (Brunauer-Emmett-Teller). The present invention further provides milnacipran hydrochloride, obtained by the process disclosed herein, is characterized by a specific surface area of about 2.19 m²/gm.

The specific surface area determination used Coulter SA3100 with following conditions described: degassing: without, sensitivity: high, calculation: BET, type: multipoint, points: 10, sample cell: 9 cm³.

The present invention provides a milnacipran hydrochloride, obtained by the process disclosed herein, is characterized by having bulk density of particles of about 0.5 g/ml. The present invention further provides milnacipran hydrochloride, obtained by the process disclosed herein, is characterized by having bulk density of particles of about 0.43 g/ml.

The bulk density used tapped density tester dual-platform ETD-1020 (Electrolab). System specifications. Speed: nominal rate of 300 taps per minute, Accuracy: Actual setting ±1 tap, Drop height: 14±2 mm. Platform rotation: 5-15 rotations/minute.

The present invention provides a milnacipran hydrochloride, obtained by the process disclosed herein, is characterized by Hausner ratio of particles of about 1.5. The present invention further provides milnacipran hydrochloride, obtained by the process disclosed herein, is characterized by Hausner ratio of particles of about 1.4.

The Hausner ratio of milnacipran hydrochloride particles can be measured using the formula: Hausner ratio is equal to tapped density divided by untapped density. Henry H. Hausner, “Fiction Conditions in a Mass of Metal Powders, “Int. J. Powder Metall. vol. 3, 1967, pp. 7-13.

The present invention provides a milnacipran hydrochloride, obtained by the process disclosed herein, is characterized by compressibility index (Carr index, CI) of particles of about 30. The present invention further provides milnacipran hydrochloride, obtained by the process disclosed herein, is characterized by compressibility index (Carr index, CI) of particles of about 25.

The compressibility index of milnacipran hydrochloride particles can be measured using the formula:

$$C I = 100 \times \frac{{V}_{B} - {V}_{T}}{{V}_{B}}$$

V_{B}: freely settled volume of a given mass of powder,
V_{T}: tapped volume of the same mass of powder.

The present invention provides a preparation of other pharmaceutically acceptable salts of milnacipran, the process comprising reacting milnacipran crystalline form-G, as starting material or as an intermediate, with pharmaceutically acceptable salts.

The pharmaceutical acceptable salts include acid addition salts formed with inorganic acids or with organic acids. The inorganic acids may be selected from hydrochloric acid, hydrobromic acid, hydroiodic acid, sulfuric acid, nitric acid, phosphoric acid, and the like; organic acids such as acetic acid, propionic acid, hexanoic acid, heptanoic acid, malonic acid, succinic acid, malic acid, malice acid, tartaric acid, citric acid, oxalic acid, and the like, preferably hydrochloric acid.

As used herein, the term “isolated” refers to a chemical state well known among pharmaceutical chemists wherein the recited pharmaceutical ingredient has been separated from the medium in which it was created into a relatively pure physical state, before it is mixed with other pharmaceutical ingredients.

As used herein, the term “solid” refers to a chemical substance having a definite shape and volume; one that is neither liquid nor gaseous, before it is mixed with other pharmaceutical ingredients.

As used herein, the phrase “pharmaceutically acceptable” refers to that which is useful in preparing a pharmaceutical composition that is generally safe, non-toxic and neither biologically nor otherwise undesirable and includes that which is acceptable for veterinary use as well as human pharmaceutical use.

The present invention further provides milnacipran in solid form or a pharmaceutically acceptable salt, obtained by the processes described herein, having relatively low content of one or more organic volatile impurities.

The present invention provides a milnacipran in solid form or a pharmaceutically acceptable salt thereof; preferably the hydrochloride salt obtained using the process of the described herein, may have a residual solvent content that is within the limits given by the International Conference on Harmonization of Technical Requirements for Registration of Pharmaceuticals for Human Use (“ICH”) guidelines. The guideline solvent level depends on the type of solvent but is not more than about 5000 ppm, or about 4000 ppm, or about 3000 ppm.

The present invention provides a milnacipran hydrochloride, obtained by the process disclosed herein, having less than about 800 parts per million (ppm) methanol, ethanol, isopropanol, preferably less than about 200 ppm; less than about 500 ppm ethyl acetate, preferably less than about 100 ppm; less than about 500 ppm acetone, preferably less than about 100 ppm; less than about 500 ppm toluene, preferably less than about 100 ppm; less than about 500 ppm cyclohexane, preferably less than about 200 ppm; less than about 500 ppm tetrahydrofuran, preferably less than about 100 ppm; less than about 250 ppm petroleum ether, preferably less than about 100 ppm; less than about 500 ppm dichloromethane, preferably less than about 100 ppm; less than about 500 ppm n-hexane, preferably less than about 100 ppm; less than about 500 ppm n-heptane, preferably less than about 100 ppm.

The present invention also encompasses a pharmaceutical composition comprising solid crystalline milnacipran form-G and a pharmaceutically acceptable salt thereof.

The present invention provides for a pharmaceutical composition comprising the milnacipran in solid crystalline form-G, obtained by the process herein described above, and the use of said pharmaceutical composition for depression and chronic pain conditions.

The present invention further provides milnacipran in solid crystalline form-G obtained by process described herein, which is stable and is well suited for use in preparing pharmaceutical formulations. The pharmaceutical formulations according to the present invention can be administered by any appropriate route, for example orally, parenterally, or intravenously, in liquids or solid form.

The present invention further provides a milnacipran in solid form, as disclosed herein for use in a pharmaceutical composition, previously described, which may independently have a D₅₀ and D₉₀ particle size less than about 300 microns, preferably less than about 200 microns, more preferably less than about 150 microns, still more preferably less than about 50 microns and most preferably less than about 10 microns. Whereupon, the notation D_{X} means that X % of particles have a diameter less than a specified diameter D. Thus, a D₅₀ of about 300 microns means that 50% of the micronized particles in a composition have a diameter less than about 300 microns. Any milling, grinding, micronizing or other particle size reduction method known in the art can be used to bring the solid state milnacipran in solid form into any desired particle size range set forth above.

The following examples are provided to enable one skilled in the art to practice the invention and are merely illustrative of the invention. The examples should not be read as limiting the scope of the invention as defined in the features and advantages.

### EXAMPLES

### Example 1

### Preparation of Milnacipran in Solid Form in Crystalline Form-G.

(Z)-1-phenyl-1-diethylaminocarbonyl-2-phthalimidomethyl cyclopropane (15 gms, 0.039 moles), toluene (105 ml) and water (15 ml) was taken in a round bottom flask at temperature 25° C. to 30° C. and cooled to 10° C. to 15° C. 40% w/w aqueous monomethylamine (30 gms, 0.387 moles) was added drop wise to the above reaction solution at temperature 10° C. to 15° C. Reaction mass was stirred at temperature 25° C. to 30° C. for 20 hours. The aqueous layer was separated and extracted with toluene (2×30 ml). The organic layer was combined with water (45 ml) and adjusted the pH to 2 with hydrochloric acid at temperature 10° C. to 15° C. The aqueous layer was separated and added toluene (75 ml) followed by adjusting the pH to 12 with 25 ml of aqueous sodium hydroxide solution (2.5 gm sodium hydroxide flakes dissolved in 25 ml of water). The organic layer was separated and concentrated completely under reduced pressure at temperature of 40° C. to 45° C. Isopropyl ether (45 ml) was added to the resultant residue and the reaction mass was stirred at 5° C. to 10° C. The resulting solid was filtered and washed with chilled isopropyl ether (5 ml). The wet product was dried at 35° C.-40° C. under reduced pressure to provide the milnacipran in solid form Yield: 7.0 gms.

HPLC purity: 99.46

IR (KBR): C═O 1621

1H NMR (DMSO) δ ppm (TMS): 0.56 (t, 3H, CH₃); 0.95 (t, 3H, CH₃); 1.2 (m, 1H, cyclopropane); 1.2-1.8 (t, 2H, cyclopropane); 1.25 (s, 2H, —NH₂); 2.3-2.6 (q, 2H, CH₂-amine); 3.0-3.5 (m, 4H, CH₂-methyl); 7.1-7.3 (m, 5H, aromatic).

### Example 2

### Preparation of Milnacipran in Solid Form in Crystalline Form-G.

(Z)-1-phenyl-1-diethylaminocarbonyl-2-phthalimidomethyl cyclopropane (15 gms, 0.039 moles), toluene (105 ml) and water (15 ml) was taken in a round bottom flask at temperature 25° C. to 30° C. and cooled to 10° C. to 15° C. 40% w/w aqueous monomethylamine (30 gms, 0.387 moles) was added drop wise to the above reaction solution at temperature 10° C. to 15° C. Reaction mass was stirred at temperature 25° C. to 30° C. for 20 hours. The aqueous layer was separated and extracted with toluene (2×30 ml). The organic layer was combined with water (45 ml) and adjusted pH to 2 with hydrochloric acid at temperature 10° C. to 15° C. The aqueous layer was separated and added toluene (75 ml) followed by adjusted pH to 12 with 25 ml of aqueous sodium hydroxide solution (2.5 gms sodium hydroxide flakes dissolved in 25 ml of water). The organic layer was separated and concentrated completely under reduced pressure at temperature 40° C. to 45° C. Cyclohexane (45 ml) was added to the resultant residue and the reaction mass were stirred at 5° C. to 10° C. The resulting solid was filtered and washed with chilled cyclohexane (5 ml). The wet product was dried at 35° C.-40° C. under reduced pressure to provide the milnacipran in solid form Yield: 6.2 gms.

HPLC purity: 97.55

IR (KBR): C═O 1621

1H NMR (DMSO) δ ppm (TMS): 0.56 (t, 3H, CH₃); 0.95 (t, 3H, CH₃); 1.2 (m, 1H, cyclopropane); 1.2-1.8 (t, 2H, cyclopropane); 1.25 (s, 2H, —NH₂); 2.3-2.6 (q, 2H, CH₂-amine); 3.0-3.5 (m, 4H, CH₂-methyl); 7.1-7.3 (m, 5H, aromatic)

### Example 3

### Preparation of Milnacipran in Solid Form in Crystalline Form-G.

(Z)-1-phenyl-1-diethylaminocarbonyl-2-phthalimidomethyl cyclopropane (15 gms, 0.039 moles), toluene (105 ml) and water (15 ml) was taken in a round bottom flask at temperature 25° C. to 30° C. and cooled to 10° C. to 15° C. 40% w/w aqueous monomethylamine (30 gms, 0.387 moles) was added drop wise to the above reaction solution at temperature 10° C. to 15° C. Reaction mass was stirred at temperature 25° C. to 30° C. for 20 hours. The aqueous layer was separated and extracted with toluene (2×30 ml). The organic layer was combined with water (45 ml) and adjusted pH to 2 with hydrochloric acid at temperature 10° C. to 15° C. The aqueous layer was separated and added toluene (75 ml) followed by adjusted pH to 12 with 27 ml of aqueous sodium hydroxide solution (2.7 gm sodium hydroxide flakes dissolved in 27 ml of water). The organic layer was separated and concentrated completely under reduced pressure at temperature 40° C. to 45° C. n-heptane (45 ml) was added to the resultant residue and the reaction mass were stirred at 5° C. to 10° C. The resulting solid was filtered and washed with chilled n-heptane (5 ml). The wet product was dried at 35° C.-40° C. under reduced pressure to provide the milnacipran in solid form Yield: 7.2 gms.

HPLC purity: 97.53

IR (KBR): C═O 1621

1H NMR (DMSO) δ ppm (TMS): 0.56 (t, 3H, CH₃); 0.95 (t, 3H, CH₃); 1.2 (m, 1H, cyclopropane); 1.2-1.8 (t, 2H, cyclopropane); 1.25 (s, 2H, —NH₂); 2.3-2.6 (q, 2H, CH₂-amine); 3.0-3.5 (m, 4H, CH₂-methyl); 7.1-7.3 (m, 5H, aromatic).

### Example 4

### Preparation of Milnacipran Hydrochloride.

Charged ethyl acetate (65 ml) and milnacipran in solid form in crystalline form-G (10 gms) in a round bottom flask. Cooled the reaction mass at temperature 15° C. to 20° C. and added 15% w/w of ethyl acetate hydrochloride (6.8 g). Heated the reaction mass at temperature 20° C. to 25° C. and stirred for 2 hours. The resulting solid was filtered and washed with ethyl acetate (20 ml). The wet product was dried at 40° C.-50° C. under reduced pressure to provide the milnacipran hydrochloride (9.15 gms).

HPLC purity: 99.5%

### Example 5

### Preparation of Milnacipran Hydrochloride.

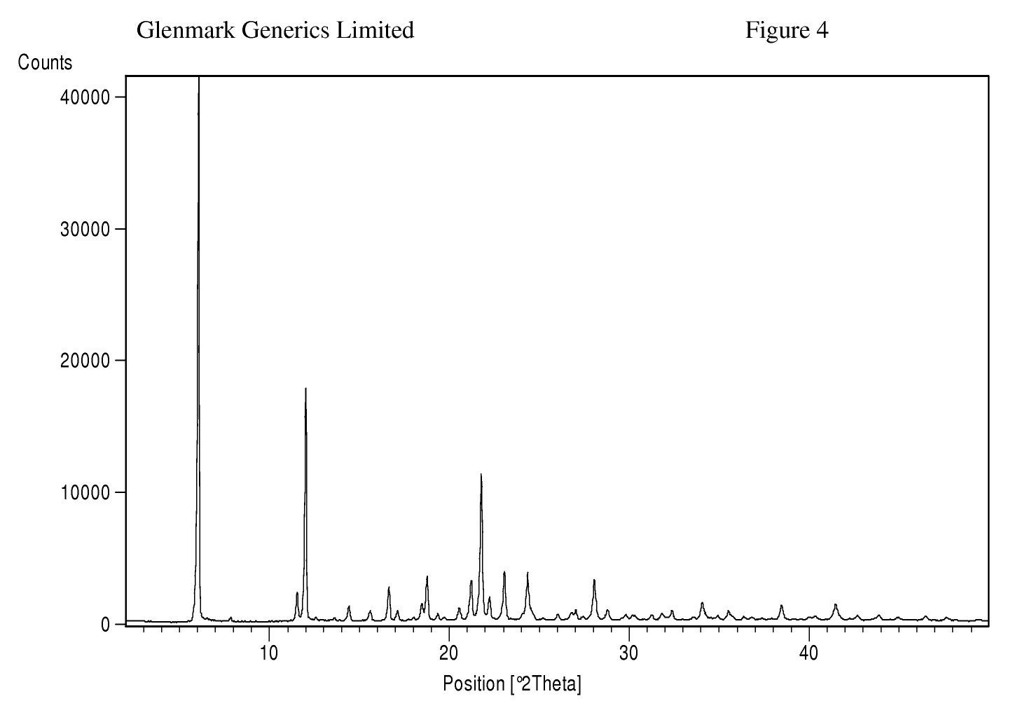

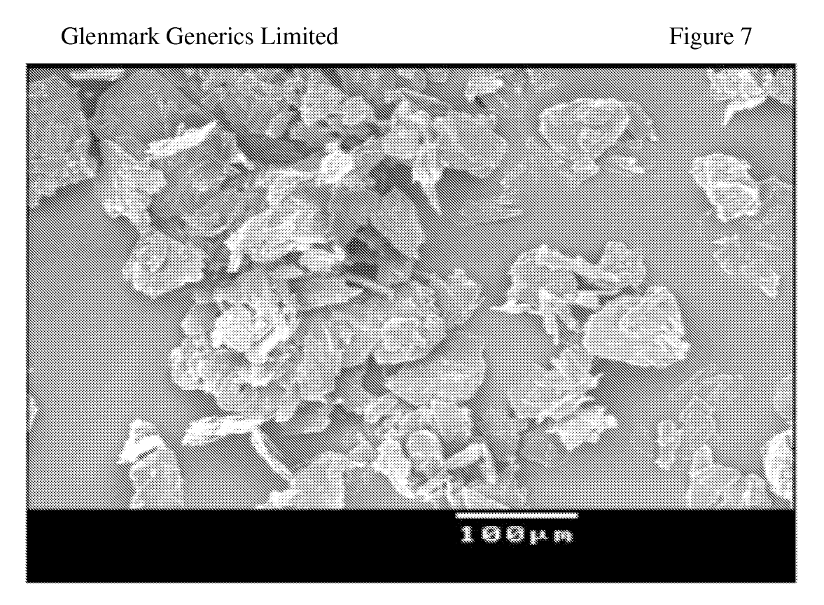

Charged ethyl acetate (60 ml), isopropyl alcohol (8 ml) and milnacipran in solid form in crystalline form-G (11.2 gms) in a round bottom flask. Cooled the reaction mass at temperature 15° C. to 20° C. and added 15% w/w of ethyl acetate hydrochloride (7.6 gm). Heated the reaction mass at temperature 20° C. to 25° C. and stirred for 3 hours. The resulting solid was filtered and washed with ethyl acetate (30 ml). The wet product was dried at 40° C.-50° C. under reduced pressure to provide the milnacipran hydrochloride (10.15 gms) and is characterized and the results are as follows:

| S. No. | Test | Results |
|---|---|---|
| 1 | HPLC purity | 99.8% |
| 2 | Specific optical rotation | +2.20° |
| 3 | XRD | As set forth in FIG. 4 |
| 4 | Identification by DSC | One endothermic peak gets at |
| | | 180.71° C. |
| 5 | TGA | 0.26% sample weight lost up to |
| | | 100° C. |
| 6 | Particle size distribution | d(0.1) 16.22 μm, d(0.5) 40.38 μm, |
| | | d(0.9) 79.38 μm |
| 7 | SEM | As set forth in FIG. 7 |
| 8 | Specific surface area by | 2.19 m²/gm |
| | BET | |
| 9 | Bulk density | Bulk density = 0.30 g/ml |
| | | Tapped density = 0.43 g/ml |
| 10 | Compressibility Index | 29.31 |
| 11 | Hausner ratio | 1.41 |

Herein, as shown above, Examples 1-3 typify the preparation of solid crystalline milnacipran. Example 1 is describing preparation of solid crystalline milnacipran from isopropyl ether, example 2 is describing preparation of solid crystalline milnacipran from cyclohexane and example 3 is describing preparation of solid crystalline milnacipran from n-heptane.

Further, Examples 4 and 5 are presentations of the typical preparation of milnacipran hydrochloride, using the solid crystalline milnacipran form-G, prepared in the process herein described. Example 4 is describing preparation of milnacipran hydrochloride from ethyl acetate and Example 5 is describing preparation of milnacipran hydrochloride from mixture of ethyl acetate and isopropyl alcohol. The resultant milnacipran hydrochloride exhibits the tabulated properties presented after Example 5.

The '836 patent and '957 publication disclose the preparation of milnacipran, but not its isolation as solid crystalline material. In contrast, the process for the preparation of milnacipran, herein described, arrives at a milnacipran in solid form, which may be isolated as a solid by a crystallization process and contains a substantially low content of formula II, or formula III, or formula IV, or formula V (see supra). Particularly, the process herein described allows that a milnacipran may be isolated as a solid, particularly in crystalline form.

The present invention encompasses methods of preparing milnacipran in solid crystalline form-G and a pharmaceutically acceptable salt thereof with high purity. The processes of the invention allow for economical synthesis, shorter reaction times, and yields of high purity.

We claim:

1. Milnacipran in solid crystalline form-G.

2. The compound of claim 1, with an XRD pattern substantially in accordance with FIG. 1 and a differential scanning calorimetry (DSC) thermogram substantially in accordance with FIG. 2.

3. The compound of claim 1, having a purity of at least about 97%, as determined by HPLC.

4. A process for the preparation of milnacipran in solid crystalline form-G, comprising
a) reacting (Z)-1-phenyl-1-diethylaminocarbonyl-2-phthalimidomethyl cyclopropane of formula II, 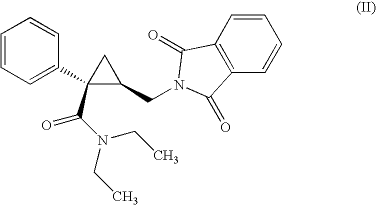
with monomethylamine in a first organic solvent to form a reaction solution,
b) isolating the milnacipran free base from the reaction solution,
c) crystallizing the milnacipran free base with a second organic solvent selected from C₁₋₄ alcohols, ketones, esters, hydrocarbons, ethers, halogenated solvents, water and their mixtures.

5. The process of claim 4, wherein the first organic solvent is selected from C₁₋₄ alcohols, hydrocarbons, halogenated solvents, esters, water and their mixtures.

6. The process of claim 4, wherein the first organic solvent is selected from methanol, ethanol, toluene, ethyl acetate, water and their mixtures.

7. The process of claim 4, wherein the first organic solvent is mixture of toluene and water.

8. The process of claim 4, wherein the isolation is done by concentrating the reaction solution.

9. The process of claim 4, wherein the second organic solvent is methanol, ethanol, isopropanol, acetone, ethyl acetate, isopropyl acetate, isopropyl ether, toluene, cyclohexane, n-pentane, n-hexane, n-heptane, dichloromethane, water and mixtures thereof.

10. The process of claim 4, wherein the step of crystallization is carried out at temperature of about −10° C. to about 10° C.

11. The process of claim 4, further comprising converting the milnacipran in solid form-G into a pharmaceutically acceptable salt thereof.

12. The process of claim 11, wherein the pharmaceutically acceptable salt is hydrochloride salt.

13. A process for the preparation of milnacipran hydrochloride, comprising:
a) providing a solution of milnacipran in solid form obtained from the process of claim 10;
b) treating the solution with hydrochloric acid,
c) isolating the milnacipran hydrochloride.

14. The process of claim 13, wherein the milnacipran hydrochloride has a purity of at least about 99.8% as determined by HPLC.

15. The process of claim 13, wherein the milnacipran hydrochloride is substantially free of 1-phenyl-1-diethylaminocarbonyl-2-phthalimidomethyl cyclopropane of formula II, as determined by HPLC.

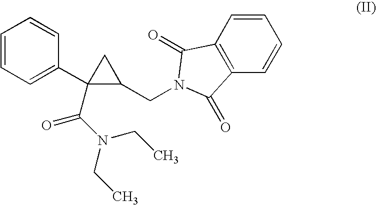

16. The process of claim 13, wherein the milnacipran hydrochloride is substantially free of 1-phenyl-1-ethylaminocarbonyl-2-phthalimidomethyl cyclopropane of formula III, as determined by HPLC.

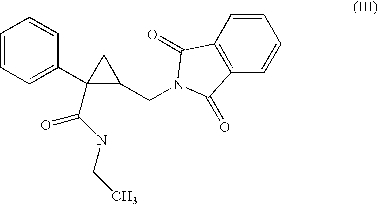

17. The process of claim 13, wherein the milnacipran hydrochloride is substantially free of 2-(aminomethyl)-N-ethyl-1-phenylcyclopropanecarboxamide of formula IV, as determined by HPLC.

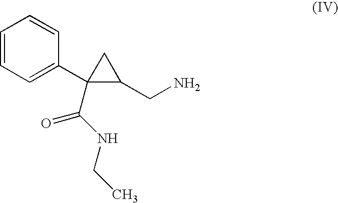

18. The process of claim 13, wherein the milnacipran hydrochloride is substantially free of trans isomer of 2-(aminomethyl)-N,N-diethyl-1-phenylcyclopropane carboxamide (trans milnacipran) of formula V, as determined by HPLC. [chemical structure]
